(19) United States
(12) Patent Application Publication
(10) Pub. No.: US 2010/0021561 A1
(43) Pub. Date: Jan. 28, 2010

(54) SELF-PRESERVED AQUEOUS PHARMACEUTICAL COMPOSITIONS

(76) Inventors: Masood A. Chowhan, Arlington, TX (US); David J. Keith, Washington, MO (US); Bahram Asgharian, Arlington, TX (US)

Correspondence Address: ALCON, IP LEGAL, TB4-8, 6201 SOUTH FREEWAY, FORT WORTH, TX, 76134, US

(21) Appl. No.: 12/441,742

(22) PCT Filed: Sep. 20, 2007
(86) PCT No.: PCT/US07/79091

§ 371 Date: Mar. 18, 2009

Related U.S. Application Data
(60) Provisional application No. 60/826,514, filed on Sep. 21, 2006.

(51) Int. Cl. A61K 33/30; A61P 31/04; A61P 27/02; A61K 31/131

(52) U.S. Cl. 424/641; 514/667

(57) ABSTRACT

The use of borate/amino alcohol/zinc systems to enhance the antimicrobial activity of multi-dose pharmaceutical compositions is described. The compositions do not require a conventional anti-microbial preservative and therefore are referred to as being “self-preserved”. The compositions possess sufficient antimicrobial activity to satisfy the preservative efficacy requirements of the USP for aqueous ophthalmic compositions.

### BACKGROUND OF THE INVENTION

The present invention is directed to self-preserved pharmaceutical compositions. More specifically, the invention is directed to the provision of aqueous, multi-dose pharmaceutical compositions that have been formulated so as to have sufficient antimicrobial activity to satisfy the preservation efficacy requirements of the United States Pharmacopeia (“USP”) and analogous guidelines in other countries, without requiring a conventional antimicrobial preservative, such as benzalkonium chloride, polyquaternium-1, hydrogen peroxide (e.g., sodium perborate), or chorine-containing agents. The ability to achieve self-preservation is based on a unique combination of formulation components and criteria.

Many pharmaceutical compositions are required to be sterile (i.e., free of bacteria, fungi and other pathogenic microorganisms). Examples of such compositions include: solutions and suspensions that are injected into the bodies of humans or other mammals; creams, lotions, solutions or other preparations that are topically applied to wounds, abrasions, burns, rashes, surgical incisions, or other conditions where the skin is not intact; and various types of compositions that are applied either directly to the eye (e.g., artificial tears, irrigating solutions, and drug products), or are applied to devices that will come into contact with the eye (e.g., contact lenses).

The foregoing types of compositions can be manufactured under sterile conditions via procedures that are well known to those skilled in the art. However, once the packaging for a product is opened, such that the composition contained therein is exposed to the atmosphere and other sources of potential microbial contamination (e.g., the hands of a human patient), the sterility of the product may be compromised. Such products are typically utilized multiple times by the patient, and are therefore frequently referred to as being of a “multi-dose” nature.

Due to the frequent, repeated exposure of multi-dose products to the risk of microbial contamination, it is necessary to employ a means for preventing such contamination from occurring. The means employed may be: (i) a chemical agent that prevents the proliferation of microbes in a composition, which is referred to herein as an “antimicrobial preservative”; or (ii) a packaging system that prevents or reduces the risk of microbes reaching a pharmaceutical composition within a container.

Prior multi-dose ophthalmic compositions have generally contained one or more antimicrobial preservatives in order to prevent the proliferation of bacteria, fungi and other microbes. Such compositions may come into contact with the cornea either directly or indirectly. The cornea is particularly sensitive to exogenous chemical agents. Consequently, in order to minimize the potential for harmful effects on the cornea, it is preferable to use anti-microbial preservatives that are relatively non-toxic to the cornea, and to use such preservatives at the lowest possible concentrations (i.e., the minimum amounts required in order to perform their anti-microbial functions).

Balancing the anti-microbial efficacy and potential toxicological effects of anti-microbial preservatives is sometimes difficult to achieve. More specifically, the concentration of an antimicrobial agent necessary for the preservation of ophthalmic formulations from microbial contamination may create the potential for toxicological effects on the cornea and/or other ophthalmic tissues. Using lower concentrations of the anti-microbial agents generally helps to reduce the potential for such toxicological effects, but the lower concentrations may be insufficient to achieve the required level of biocidal efficacy (i.e., antimicrobial preservation).

The use of an inadequate level of antimicrobial preservation may create the potential for microbial contamination of the compositions and ophthalmic infections resulting from such contaminations. This is also a serious problem, since ophthalmic infections involving Pseudomonas aeruginosa or other virulent microorganisms can lead to loss of visual function or even loss of the eye.

Thus, there is a need for a means of enhancing the activity of anti-microbial agents so that very low concentrations of the agents can be utilized without increasing the potential for toxicological effects or subjecting patients to unacceptable risks of microbial contamination and resulting ophthalmic infections.

Ophthalmic compositions are generally formulated as isotonic, buffered solutions. One approach to enhancing the anti-microbial activity of such compositions is to include multi-functional components in the compositions. In addition to performing their primary functions, these multi-functional components also serve to enhance the overall anti-microbial activity of the compositions.

The following publications may be referred to for further background regarding the use of multi-functional components to enhance the antimicrobial activity of ophthalmic compositions:
1. U.S. Pat. No. 5,817,277 (Mowrey-McKee, et al; tromethamine);
2. U.S. Pat. No. 6,503,497 (Chowhan, et al.; borate/polyol complexes);
3. U.S. Pat. No. 5,741,817 (Chowhan, et al.; low molecular weight amino acids such as glycine);
4. U.S. Pat. No. 6,319,464 (Asgharian; low molecular weight amino alcohols);
5. U.S. Patent Application Publication No. US 2002/0122831 A1 (Mowrey-McKee, et al.; bis-aminopolyols);
6. U.S. Pat. No. 6,348,190 (Illes, et al.; zinc); and
7. JP 2003-104870 (zinc).

The compositions of the present invention are multi-dose products that do not contain a conventional antimicrobial preservative (e.g., benzalkonium chloride), but yet are preserved from microbial contamination. Such compositions have been referred to in the art as being “preservative free” (see, e.g., U.S. Pat. No. 5,597,559 issued to Olejnik, et al.). Compositions that are preserved from microbial contamination as a result of the inherent antimicrobial activity of one or more components of the compositions are also referred to in the art as being “self-preserved” (see, e.g., U.S. Pat. No. 6,492,361 issued to Muller, et al.).

The following publication may be referred to for further background regarding pharmaceutical compositions that are “preservative-free” or “self-preserving”: Kabara, et al., Preservative-Free and Self-Preserving Cosmetics and Drugs—Principles and Practice, Chapter 1, pages 1-14, Marcel Dekker, Inc. (1997).

The multi-dose compositions of the present invention, which do not contain a conventional antimicrobial preservative, are referred to herein as being “self-preserved”.

### SUMMARY OF THE INVENTION

The present invention is based on a finding that by combining certain formulation components and maintaining specific formulation parameters, it is possible to create aqueous, multi-dose compositions that satisfy the preservative efficacy requirements of the USP without a conventional antimicrobial preservative. It is understood that there are a number of ingredients which exert antimicrobial effect and are not conventional preservatives. The ingredients disclosed here are (1) ingredients used in pharmaceutical preparations and (2) have limited antimicrobial effect at the concentrations used by themselves. The formulation components include: (i) borate, preferably in combination with one or more polyols; (ii) one or more amino alcohols; and (iii) zinc ions. The compositions are preferably formulated to have a pH of 7.6 or higher. The most preferred pH for ophthalmic compositions is 7.8-8.2. As explained below, it has been discovered that a pH in this range is desirable in order to maximize the antimicrobial activity of the borate/amino alcohol systems described herein.

The use of borate-polyol complexes to enhance antimicrobial activity is described in U.S. Pat. No. 6,503,497 (Chowhan, et al.), and the use of low molecular weight amino alcohols to enhance antimicrobial activity is described in U.S. Pat. No. 6,319,464 (Asgharian). The entire contents of both of these patents are hereby incorporated in the present specification by reference.

U.S. Pat. No. 6,319,464 teaches that the amino alcohols described therein should preferably be utilized in combination with a borate or borate/polyol buffer system (see last paragraph in column 4). The '464 patent also teaches that the antimicrobial effect of the amino alcohol/borate systems described therein may eliminate the need for a conventional preservative agent (see lines 39-43 in column 3). However, the above-cited '464 and '497 patents do not disclose or suggest that pH is a critical factor relative to achieving a level of antimicrobial activity that is sufficient in order to produce aqueous, multi-dose ophthalmic compositions that have adequate antimicrobial activity to satisfy the preservative efficacy standards of the United States Pharmacopeia (i.e., the Antimicrobial Efficacy Test described in USP 24, USP 25, USP 26 and USP 27), without a conventional antimicrobial preservative.

The present invention is based in-part on a finding that a pH of 7.6 or higher is critical in order to maximize the antimicrobial activity of borate/amino alcohol systems. As a result of this finding, the present inventors have produced aqueous, multi-dose ophthalmic compositions that satisfy the USP preservative efficacy standards for ophthalmic solutions without employing a conventional antimicrobial preservative, such as benzalkonium chloride or polyquaternium-1.

The present invention is also based in-part on a finding that zinc further enhances the antimicrobial activity of the borate/amino alcohol systems described herein. The use of zinc to enhance the antimicrobial activity of pharmaceutical compositions, including ophthalmic solutions, is well known. See, for example, the following articles and patent publications, as well as U.S. Pat. No. 6,348,190 and JP 2003-104870, cited above:
McCarthy, “Metal Ions and Microbial Inhibitors”, Cosmetic & Toiletries, 100:69-72 (February 1985);
Zeelie, et al., “The Effects of Selected Metal Salts on the Microbial Activities of Agents used in the Pharmaceutical and Related Industries”, Metal Compounds in Environment and Life, 4:193-200 (1992);
Zeelie, et al., “Effects of Copper and Zinc Ions on the Germicidal Properties of Two Popular Pharmaceutical Antiseptic Agents, Cetylpyridinium Chloride and Povidone-iodine”, Analyst, 123:503-507 (March 1998);
McCarthy, et al., “The Effect of Zinc Ions on the Antimicrobial Activity of Selected Preservatives”, Journal of Pharmacy and Pharmacology, Vol. 41 (1989);
U.S. Pat. No. 6,482,799 (Tuśe, et al.);
U.S. Pat. No. 5,320,843 (Raheja, et al.);
U.S. Pat. No. 5,221,664 (Berkowitz, et al.);
U.S. Pat. No. 6,034,043 (Fujiwara, et al.);
U.S. Pat. No. 4,522,806 (Muhlemann, et al.);
U.S. Pat. No. 6,017,861 (Fujiwara, et al.); and
U.S. Pat. No. 6,121,315 (Nair, et al.).
   However, the use of zinc ions in combination with borate and amino alcohol systems of the type described herein is not disclosed or suggested by the prior art.

The self-preserved, multi-dose compositions of the present invention have several advantages over existing ophthalmic formulations that are either: (i) packaged as a “single dose” or “unit of use” product, so as to avoid the inclusion of any antimicrobial preservative (e.g., BION® TEARS Lubricant Eye props, which is marketed by Alcon Laboratories, Inc.), or (ii) preserved by means of a so-called “disappearing” preservatives, such as the chlorite-based system described in U.S. Pat. Nos. 5,424,078; 5,736,165; 6,024,954; and 5,858,346 (e.g., the artificial tears product “REFRESH™ Tears”, which is marketed by Allergan), or the peroxide-containing system described in U.S. Pat. Nos. 5,607,698; 5,683,993; 5,725,887; and 5,858,996 (e.g., the artificial tear product “GenTeal™ Tears”, which is marketed by CIBAVision).

Unlike these existing products, the multi-dose ophthalmic compositions of the present invention are able to satisfy the USP preservative efficacy requirements without employing any conventional antimicrobial preservatives, such as chlorite or hydrogen peroxide.

The above-discussed findings regarding the role of pH and zinc chloride may be applied to enhance the antimicrobial activity of various types of pharmaceutical compositions. However, the present invention is particularly directed to the provision of aqueous ophthalmic solutions that are effective in preventing microbial contamination in the absence of conventional antimicrobial preservatives, such as benzalkonium chloride (“BAC”), polyquaternium-1, chlorite or hydrogen peroxide.

### DETAILED DESCRIPTION OF THE INVENTION

The compositions of the present invention contain a borate/amino alcohol system in an amount sufficient to enhance the antimicrobial activity of the compositions. The compositions are preferably formulated to have a pH of 7.6 or higher. A pH of 7.8-8.2 is most preferred for ophthalmic compositions.

The present invention is particularly directed to the provision of multi-dose, self-preserved ophthalmic compositions that contain a borate/amino alcohol system in an amount sufficient to allow the compositions to satisfy the USP preservative efficacy requirements, as well as other preservative efficacy standards for aqueous pharmaceutical compositions, without a conventional antimicrobial preservative.

Relative to bacteria, the USP 27 Antimicrobial Effectiveness Test requires that multi-dose ophthalmic compositions have sufficient antimicrobial activity to reduce an initial inoculum of approximately 10⁵ to 10⁶ bacteria by one log(i.e., a 90% reduction in the microorganism population) over a period of seven (7) days and by three logs (i.e., a 99.9% reduction in the microorganism population) over a period of fourteen (14) days, and requires that there cannot be any increase in the microorganism population following the conclusion of the fourteen day period. Relative to fungi, the USP standards require that the compositions maintain stasis (i.e., no growth) relative to the population of the initial inoculum over the entire 28 day test period. The margin of error in calculating microorganism populations is generally accepted to be 0.5 logs. Accordingly, the term “stasis” as utilized relative to the above-discussed USP standards means that the initial fungi population cannot increase by more than 0.5 log orders, relative to the initial population.

The preservative efficacy standards for multi-dose ophthalmic solutions in the U.S. and other countries/regions are set forth in the following table:

Preservative Efficacy Test (“PET”) Criteria
(Log Order Reduction of Microbial Inoculum Over Time

| | Bacteria | Fungi |
|---|---|---|
| USP 27 | A reduction of 1 log (90%), | The compositions must demonstrate |
| | by day 7; 3 logs (99.9%) by | stasis over the entire test period, which |
| | day 14; and no increase after | means no increases of 0.5 logs or greater, |
| | day 14 | relative to the initial inoculum. |
| Japan | A reduction of 3 logs | No increase from initial count at 14 and |
| | (99.9%) by 14 days; and no | 28 days |
| | increase from day 14 | |
| | through day 28. | |
| Ph. Eur. A¹ | A reduction of 2 logs (99%) | A reduction of 2 logs (99%) by 7 days, |
| | by 6 hours; 3 logs by 24 | and no increase thereafter |
| | hours; and no recovery after | |
| | 28 days | |
| Ph. Eur. B | A reduction of 1 log at 24 | A reduction of 1 log (90%) by day 14, |
| | hours; 3 logs by day 7; and | and no increase thereafter |
| | no increase thereafter | |
| FDA/ISO | A reduction of 3 logs from | No increase higher than the initial value |
| 14730 | initial challenge at day 14; | at day 14, and no increase higher than the |
| | and a reduction of 3 logs | day 14 rechallenge count through day 28. |
| | from rechallenge | |

¹There are two preservative efficacy standards in the European Pharmacopoeia - “A” and “B”.

The standards identified above for the USP 27 are substantially identical to the requirements set forth in prior editions of the USP, particularly USP 24, USP 25 and USP 26.

As used herein, the term “borate” includes boric acid, sodium borate and potassium borate. The use of borates containing divalent cations (e.g., calcium borate) may adversely affect the antimicrobial action of zinc ions, by competing with zinc binding sites on the cell walls of bacterial and other microbes, and is therefore not preferred. For the same reason, the self-preserved compositions of the present invention are preferably free of or substantially free of other sources of divalent cations, such as calcium chloride.

The compositions of the present invention preferably contain one or more polyols. The polyols can be linear or cyclic, substituted or unsubstituted, or mixtures thereof, so long as the resultant complex is water soluble and pharmaceutically acceptable. Examples of such compounds include: sugars, sugar alcohols, sugar acids and uronic acids. Preferred polyols are sugars, sugar alcohols and sugar acids, including, but not limited to: sorbitol, mannitol, glycerin, and xylitol. The use of sorbitol is particularly preferred.

The self-preserved compositions of the present invention preferably contain one or more borates in an amount of from about 0.1 to about 2.0% w/v, more preferably 0.3 to 1.5% w/v, and most preferably 0.5 to 1.2% w/v. The compositions of the present invention preferably contain one or more polyols in an amount of from about 0.01 to about 5.0% w/v, more preferably 0.6 to 2.0% w/v.

The low molecular weight amino alcohols which may be utilized in the present invention are water-soluble and have a molecular weight in the range of from about 60 to about 200. The following compounds are representative of the low molecular weight amino alcohols which may be utilized in the present invention: 2-amino-2-methyl-1-propanol (AMP), 2-dimethylamino-methyl-1-propanol (DMAMP), 2-amino-2-ethyl-1,3-propanediol (AEPD), 2-amino-2-methyl-1,3-propanediol (AMPD), 2-amino-1-butanol (AB). “AMP (95%)”, which refers to 95% pure AMP and 5% water, is the most preferred low molecular weight amino alcohol of the present invention. These amino alcohols are available commercially from Angus Chemical Company (Buffalo Grove, Ill.).

The amount of amino alcohol used will depend on the molecular weight of the amino alcohol selected, and the presence (or absence) of other ingredients in the composition (e.g., buffering agents and/or tonicity agents). The amino alcohol will generally be present in an amount necessary to enhance the antimicrobial activity of an aqueous self-preserved pharmaceutical composition of the type described herein. The amount of amino alcohol required for a particular composition can be determined through comparative testing, such as the tests described in Example 6 hereof. The above-described amino alcohols are also utilized in the compositions of the present invention to neutralize the pH of the borate or borate/polyol complex, or bring the composition to the desired pH level. The amount of amino alcohol required for this purpose is a function of the particular borate or borate/polyol mixture selected and the concentration thereof. In general, the self-preserved compositions of the present invention will contain one or more amino alcohols at a total concentration of from about 0.01 to about 2.0 percent by weight/volume (“% w/v”), and preferably from 0.1 to 1.0% w/v.

The compositions of the present invention preferably also contain an amount of zinc sufficient to enhance the antimicrobial activity of the borate/amino alcohol system described above. The zinc may be provided in various forms, such as zinc chloride, zinc sulfate, zinc acetate or zinc carbonate. The use of zinc chloride is preferred. The amount of zinc chloride required to achieve this effect may vary somewhat from formulation to formulation, depending on the particular borate or borate/polyol complex and amino alcohol selected, but will generally be from about 0.0005% to about 0.005% w/v, preferably 0.00075 to 0.0025% w/v.

In general, the self-preserved compositions of the present invention will preferably contain zinc, either in the form of zinc chloride or other zinc salts, at a molar concentration of 0.000017 moles/liter to 0.00017 moles/liter, preferably 0.000026 moles/liter to 0.00009 moles/liter. However, the concentration of zinc may be as high as 0.0035 moles/liter.

The manner in which zinc enhances antimicrobial activity in the compositions of the present invention is not completely understood. However, it is believed that zinc atoms enhance the antimicrobial activity of the borate/amino alcohol systems by forming bridges between the borate groups.

The borate/amino alcohol system described herein may be included in various types of pharmaceutical compositions to enhance anti-microbial activity and self-preserve the compositions, such as ophthalmic, otic, nasal and dermatological compositions, but are particularly useful in ophthalmic compositions. Examples of such compositions include: ophthalmic pharmaceutical compositions, such as topical compositions used in the treatment of glaucoma, infections, allergies or inflammation; compositions for treating contact lenses, such as cleaning products and products for enhancing the ocular comfort of patients wearing contact lenses; and various other types of ophthalmic compositions, such as ocular lubricating products, artificial tears, astringents, and so on. The compositions may be aqueous or non-aqueous, but will generally be aqueous.

The present invention is particularly directed to the provision of self-preserved, multi-dose ophthalmic compositions in connection with the treatment of conditions wherein the cornea or adjacent ocular tissues are irritated, or conditions requiring frequent application of a composition, such as in the treatment of dry eye patients. The self-preserved compositions of the present invention are therefore particularly useful in the field of artificial tears, ocular lubricants, contact lens rewetting drops, and other compositions used to treat dry eye conditions, as well as other conditions involving ocular inflammation or discomfort.

The ophthalmic compositions of the present invention may be formulated to include one or more agents to enhance ocular comfort and/or retention of the compositions on the eye following topical application. The types of agents which may be utilized include: cellulose derivatives, such as hydroxypropyl methylcellulose (“HPMC”); Dextran 70; polyethylene glycol; propylene glycol; carboxy vinyl polymers; polyvinyl alcohol polymers or copolymers; and polysaccharides. The preferred polysaccharides are hydroxypropyl guar and other galactomannan polymers described in U.S. Pat. No. 6,583,125 (Asgharian). The entire contents of the '125 patent are hereby incorporated in the present specification by reference.

Some of the agents described in the preceding paragraph (e.g., hydroxypropyl guar, referred to hereinafter as “hp-guar”) are capable of forming crosslinks with borate and consequently form gels at elevated pH. The formation of such gels may hamper the dispensability of the composition, particularly from a droptainer. In the event such interference is encountered, polyols such as sorbitol can be added to minimize guar crosslinks with borates. It has been found that the concentration of polyols can be adjusted to maintain some borate crosslinking of the galactomannan to provide the desired increase in ocular retention and enhanced lubricity. Another desirable attribute of polyol addition is to increase the buffer capacity of the composition. Borates and polyols provide buffering over a wide pH range of 3-9 that is particularly useful for disclosed compositions that are effective over a narrow pH range.

The compositions of the present invention will generally be formulated as sterile aqueous solutions. The compositions of the present invention will be formulated so as to be compatible with the eye and/or other tissues to be treated with the compositions (e.g., otic and nasal tissues). The ophthalmic compositions intended for direct application to the eye will be formulated so as to have a pH and tonicity which are compatible with the eye. This will normally require a buffer to maintain the pH of the composition at or near the pH levels discussed above (i.e., greater than 7.6, preferably 7.8 to 8.2), and may require a tonicity agent to bring the osmolality of the composition to a level at or near 210-350 milliosmoles per kilogram (mOsm/kg).

The following examples are presented to further illustrate selected embodiments of the present invention for artificial tear compositions.

### Example 1

The formulation shown in Table 1 below was prepared to evaluate the effect of a pH of 7.9 on the antimicrobial activity of the formulation.

TABLE 1

| | FID 103777 |
|---|---|
| | Lot Number 17110-01 |
| Component | Concentration (w/v %) |
| Dextran 70 | 0.1 |
| HPMC | 0.3 |
| Propylene Glycol | 0.3 |
| Boric acid | 0.8 |
| Sorbitol | 1.4 |
| Sodium chloride | 0.1 |
| Potassium chloride | 0.12 |
| Calcium chloride | 0.0053 |
| Magnesium chloride | 0.0064 |
| Zinc chloride | 0.00015 |
| AMP (95%) | 0.588 |
| pH | 7.9 |

The formulation described in Table 1 was prepared as follows:

### HPMC Solution:

1. In a 250 mL Pyrex media bottle, add the correct amount of 2% HPMC stock solution.
2. Autoclave at 121° C. for 30 minutes.
3. Hold the autoclaved solution for later compounding.

### Buffer Vehicle:

1. In a 250 mL beaker, add the remaining formulation chemicals for a 200 mL batch using only 150 mL of purified water.
2. Measure the pH and adjust to 7.9 with NaOH/HCl.
3. QS to 100% (150 mL) with purified water.
4. Filter the solution using a 0.2 μm CA filter unit.

### Final Formulation:

1. Slowly add the filtered buffer vehicle to the autoclaved HPMC stock solution.
2. Allow the solution to mix well.

The antimicrobial activity of the above-described solution was evaluated by means of a standard microbiological analysis (i.e., USP26 Antimicrobial Effectiveness Test). The test samples were challenged with standardized suspensions of five microorganisms, and the number of surviving microorganisms was determined at 7, 14 and 28 days. The results are presented in Table 2 below:

TABLE 2

| | Time | Log₁₀ Reduction of Survivors |
|---|---|---|
| Microorganism | (days) | Lot Number 17110-01 |
| A. niger | 7 | 2.0 |
| | 14 | 2.1 |
| | 28 | 2.9 |
| C. albicans | 7 | 0.4 |
| | 14 | 1.4 |
| | 28 | 3.0 |
| E. coli | 7 | 2.2 |
| | 14 | 5.1 |
| | 28 | 5.1 |
| P. aeruginosa | 7 | 2.5 |
| | 14 | 5.0 |
| | 28 | 5.0 |
| S. aureus | 7 | 2.1 |
| | 14 | 4.6 |
| | 28 | 4.8 |

The results demonstrate overall preservative efficacy against the organisms tested.

### Example 2

As explained above, polymers that are capable of forming complexes with borates (e.g., guar or hp-guar) have been found to reduce the antimicrobial activity of the borate/amino alcohol systems described herein. The formulation shown in Table 3 below is similar to the formulation described in Example 1, except that Dextran 70 and HPMC have been replaced by hp-guar.

A formulation nearly identical to the one shown in Table 3 was evaluated to determine if it had adequate antimicrobial activity to satisfy USP preservative efficacy requirements. It was determined that inclusion of hp-guar prevented the formulation from consistently satisfying the USP preservative efficacy requirements. However, it was unexpectedly discovered that this problem could be overcome by increasing the concentration of zinc chloride by a factor of 10 (i.e., from 0.00015 to 0.0015 w/v %). The formulation shown in Table 3, which contains this higher concentration of zinc chloride, has consistently satisfied the USP preservative efficacy requirements. The preservative efficacy test (“PET”) results for four different lots are provided below:

TABLE 3
Formulation Number FID 105783

| Component | Concentration (w/v %) |
|---|---|
| HP-Guar | 0.16 |
| Boric Acid | 0.7 |
| Sorbitol | 1.4 |
| PEG-400 | 0.4 |
| Propylene Glycol | 0.3 |
| Potassium Chloride | 0.12 |
| Sodium Chloride | 0.1 |
| Calcium Chloride | 0.0053 |
| Magnisium Chloride | 0.0064 |
| Zinc Chloride | 0.0015 |
| AMP (95%) | 0.57 |
| Hydrochloric Acid | Adj. pH |
| Target pH | 7.9 |
| Purified Water | QS to 100% |
| Volume to make (L) | 1 |

PET Results

| | Lot Number | | | |
|---|---|---|---|---|
| | PD Lot | 03-34508 | 03-34433 | 03-34632 |
| P. aeruginosa (Day 7) | 5.0 | 5.0 | 4.8 | 5.0 |
| E. coli (Day 7) | 5.0 | 5.0 | 4.9 | 5.0 |
| P. aeruginosa (Day 14) | 5.0 | 5.0 | 4.8 | 5.0 |
| E. coli (Day 14) | 5.0 | 5.0 | 4.9 | 5.0 |
| P. aeruginosa (Day 28) | 3.9* | 3.9* | 4.8 | ND |
| E. coli (Day 28) | 4.0* | 4.0* | 4.9 | ND |

*Rechallenge on day 14
**ND = Not Performed

### Example 3

The formulations shown in Tables 4 and 5 below were prepared and tested in order to evaluate the effect of small variations in pH on the antimicrobial activity of the compositions.

TABLE 4
Effect of pH

| | Formulation Numbers/Concentrations (w/v %) | | | |
|---|---|---|---|---|
| | FID | FID | | |
| | 105784 | 105801 | FID 105802 | FID 105782 |
| | Batch/Lot | | | |
| Component | 03-34662 | 03-34667 | 03-34669 | 03-34648 |
| HP-Guar | 0.16 | 0.16 | 0.16 | 0.16 |
| Boric Acid | 0.7 | 0.7 | 0.7 | 0.7 |
| Sorbitol | 1.4 | 1.4 | 1.4 | 1.4 |
| PEG-400 | 0.4 | 0.4 | 0.4 | 0.4 |
| Propylene Glycol | 0.3 | 0.3 | 0.3 | 0.3 |
| Potassium Chloride | 0.12 | 0.12 | 0.12 | 0.12 |
| Sodium Chloride | 0.1 | 0.1 | 0.1 | 0.1 |
| Calcium Chloride | 0.0053 | 0.0053 | 0.0053 | 0.0053 |
| Magnisium Chloride | 0.0064 | 0.0064 | 0.0064 | 0.0064 |
| Zinc Chloride | 0.00075 | 0.00075 | 0.00075 | 0.00075 |
| AMP (95%) | 0.6 | 0.6 | 0.6 | 0.6 |
| Hydrochloric Acid | Adj. pH | Adj. pH | Adj. pH | Adj. pH |
| Target pH | 7.0 | 7.3 | 7.6 | 7.9 |
| Purified Water | QS to | QS to | QS to 100% | QS to 100% |
| | 100% | 100% | | |
| Volume to make (L) | 1 | 1 | 1 | 1 |
| PET Results (Day 7) | | | | |
| P. aeruginosa | −0.5 | −0.6 | −0.2 | 2.1 |
| E. coli | −0.5 | 0.1 | 3.3 | 5.0 |

The results presented in Table 4 show that as the pH of the formula is increased, the activity against the test organisms consistently improved. At a pH of 7.9, the composition satisfied the USP 26 preservative efficacy requirements. However, the compositions having a pH of less than 7.9 did not have adequate antimicrobial activity to satisfy the USP requirements.

The antimicrobial activities of two formulations that were identical except for pH were also compared. As shown in Table 5 below, the formulation having a pH of 7.7 did not satisfy USP 26 preservative efficacy requirements, but the formulation having a pH of 7.9 did meet those requirements.

TABLE 5
Effect of pH

| Component | Concentration (w/v %) | Concentration (w/v %) |
|---|---|---|
| HP-Guar | 0.16 | 0.16 |
| Boric Acid | 0.7 | 0.7 |
| Sorbitol | 1.4 | 1.4 |
| PEG-400 | 0.4 | 0.4 |
| Propylene Glycol | 0.3 | 0.3 |
| Potassium | 0.12 | 0.12 |
| Chloride | | |
| Sodium Chloride | 0.1 | 0.1 |
| Calcium Chloride | 0.0053 | 0.0053 |
| Magnesium | 0.0064 | 0.0064 |
| Chloride | | |
| Zinc Chloride | 0.0015 | 0.0015 |
| AMP (95%) | 0.6 | 0.6 |
| HCl/Adjust pH to | 7.9 | 7.7 |
| Purified Water | QS 100 | QS 100 |
| Microbiology | Passes USP | Fails USP |

### Example 4

The formulations shown in Table 6 below were prepared in order to evaluate the effect of zinc chloride on antimicrobial activity. The first two solutions, which contained no zinc and 1.5 ppm of zinc chloride, respectively, did not satisfy the USP 26 preservative efficacy requirements, but the third solution, which contained 15 ppm of zinc chloride, did meet those requirements.

TABLE 6
Effect of Zinc Level

| | Formulation Numbers/Concentrations (w/v %) | | |
|---|---|---|---|
| | FID 105689 | FID 104706 | FID 105688 |
| | Batch/Lot | | |
| Component | 03-34434 | 03-34405 | 03-34433 |
| HP-Guar | 0.16 | 0.16 | 0.16 |
| Boric Acid | 0.7 | 0.7 | 0.7 |
| PEG-400 | 0.4 | 0.4 | 0.4 |
| Propylene Glycol | 0.3 | 0.3 | 0.3 |
| Sorbitol | 1.4 | 1.4 | 1.4 |
| Sodium Chloride | 0.1 | 0.1 | 0.1 |
| Potassium Chloride | 0.12 | 0.12 | 0.12 |
| Calcium Chloride | 0 | 0.0053 | 0.0053 |
| Magnisium Chloride | 0 | 0.0064 | 0.0064 |
| Zinc Chloride | 0 | 0.00015 | 0.0015 |
| AMP (95%) | 0.6 | 0.6 | 0.6 |
| Hydrochloric Acid | Adj. pH | Adj. pH | Adj. pH |
| Target pH | 7.9 | 7.9 | 7.9 |
| Purified Water | QS to 100% | QS to 100% | QS to 100% |
| PET Results (Day 7) | | | |
| P. aeruginosa | 2.6 | 0.7 | 4.8 |
| E. coli | 0.9 | 1.8 | 4.9 |

### Example 5

The effect of zinc chloride on antimicrobial activity was further investigated by evaluating the preservative efficacy of the solutions shown in Table 7 below. The zinc chloride concentrations evaluated were 1.5 ppm, 3.0 ppm, 3.5 ppm, 7.5 ppm and 15 ppm, respectively. The results presented at the bottom of Table 7 show greater antimicrobial activity with increasing concentrations of zinc chloride. At 15 ppm, the two test organisms were totally eliminated (i.e., no survivors).

TABLE 7
Effect of Zinc Levels

| | Formulation Number/Concentration (w/v %) | | | | |
|---|---|---|---|---|---|
| | FID 104706 | FID 105780 | FID 105792 | FID 105782 | FID 105783 |
| | Batch/Lot | | | | |
| Component | 03-34628 | 03-34629 | 03-34652 | 03-34648 | 03-34632 |
| HP-Guar | 0.16 | 0.16 | 0.16 | 0.16 | 0.16 |
| Boric Acid | 0.7 | 0.7 | 0.7 | 0.7 | 0.7 |
| Sorbitol | 1.4 | 1.4 | 1.4 | 1.4 | 1.4 |
| PEG-400 | 0.4 | 0.4 | 0.4 | 0.4 | 0.4 |
| Propylene Glycol | 0.3 | 0.3 | 0.3 | 0.3 | 0.3 |
| Potassium Chloride | 0.12 | 0.12 | 0.12 | 0.12 | 0.12 |
| Sodium Chloride | 0.1 | 0.1 | 0.1 | 0.1 | 0.1 |
| Calcium Chloride | 0.0053 | 0.0053 | 0.0053 | 0.0053 | 0.0053 |
| Magnisium Chloride | 0.0064 | 0.0064 | 0.0064 | 0.0064 | 0.0064 |
| Zinc Chloride | 0.00015 | 0.0003 | 0.00045 | 0.00075 | 0.0015 |
| AMP (95%) | 0.6 | 0.6 | 0.6 | 0.6 | 0.6 |
| Hydrochloric Acid | Adj. pH | Adj. pH | Adj. pH | Adj. pH | Adj. pH |
| Target pH | 7.9 | 7.9 | 7.9 | 7.9 | 7.9 |
| Purified Water | QS to 100% | QS to 100% | QS to 100% | QS to 100% | QS to 100% |
| PET Results (Day 7) | | | | | |
| P. aeruginosa | 1.4 | 1.5 | 1.3 | 2.1 | 5.0 |
| E. coli | 1.0 | 2.1 | 3.9 | 5.0 | 5.0 |

### Example 6

The role of amino alcohol concentration relative to antimicrobial activity was also investigated. The formulations shown in Table 8 below, which were identical except for the concentration of the amino alcohol AMP (95%), were utilized in this evaluation. As shown at the bottom of Table 8, the solutions containing AMP (95%) at concentrations of 0.2 and 0.4 w/v % did not satisfy the USP 26 preservative efficacy requirements against Pseudomonas aeruginosa, but the solution containing AMP (95%) at a concentration of 0.6 w/v % did meet those requirements.

TABLE 8
Amino Alcohol Concentration

| | Formulation Number/Concentration (w/v %) | | |
|---|---|---|---|
| | FID 105799 | FID 105800 | FID 105782 |
| | Batch/Lot | | |
| | 03-34665 | 03-34666 | 03-34648 |
| Component | Conc. (%) | Conc. (%) | Conc. (%) |
| HP-Guar | 0.16 | 0.16 | 0.16 |
| Boric Acid | 0.7 | 0.7 | 0.7 |
| Sorbitol | 1.4 | 1.4 | 1.4 |
| PEG-400 | 0.4 | 0.4 | 0.4 |
| Propylene Glycol | 0.3 | 0.3 | 0.3 |
| Potassium Chloride | 0.12 | 0.12 | 0.12 |
| Sodium Chloride | 0.1 | 0.1 | 0.1 |
| Calcium Chloride | 0.0053 | 0.0053 | 0.0053 |
| Magnisium Chloride | 0.0064 | 0.0064 | 0.0064 |
| Zinc Chloride | 0.00075 | 0.00075 | 0.00075 |
| AMP (95%) | 0.2 | 0.4 | 0.6 |
| Hydrochloric Acid | Adj. pH | Adj. pH | Adj. pH |
| Target pH | 7.9 | 7.9 | 7.9 |
| Purified Water | QS to 100% | QS to 100% | QS to 100% |
| Volume to make (L) | 1 | 1 | 1 |
| PET Results (Day 7) | | | |
| P. aeruginosa | −0.7 | −0.2 | 2.1 |
| E. coli | 5.0 | 4.9 | 5.0 |

We claim:

1. A multi-dose, self-preserved pharmaceutical composition, said composition comprising an antimicrobial effective amount of a borate/amino alcohol system and an amount of zinc ions effective to enhance the antimicrobial activity of said system wherein the composition does not contain a conventional antimicrobial preservative.

2. A composition according to claim 1, wherein the composition is an aqueous ophthalmic solution having sufficient antimicrobial activity to satisfy USP/FDA/ISO preservative efficacy requirements.

3. A method of enhancing the antimicrobial activity of a pharmaceutical composition, which comprises including in the composition an antimicrobial effective amount of a borate/amino alcohol system and an amount of zinc ions effective to enhance the antimicrobial activity of said borate/amino alcohol system wherein the composition does not contain a conventional antimicrobial preservative.

4. A method according to claim 3, wherein the composition is a multi-dose, self-preserved ophthalmic solution having sufficient antimicrobial activity to satisfy USP/FDA/ISO preservative efficacy requirements.
